SEQUENCE LISTING (6 sequences)

SEQ ID NO: 1 - protein, 13 aa, Artificial Sequence
  Synthesized peptide P1
FKTTDQQTRTTVA

SEQ ID NO: 2 - protein, 8 aa, Artificial Sequence
  Synthesized peptide P2
NPKLVTIV

SEQ ID NO: 3 - protein, 9 aa, Artificial Sequence
  Synthesized peptide P3
TVTNPARIA

SEQ ID NO: 4 - protein, 7 aa, Artificial Sequence
  Synthesized peptide P4
PAGELHP

SEQ ID NO: 5 - protein, 11 aa, Artificial Sequence
  Synthesized peptide P5
LDSPSEGRAPG

SEQ ID NO: 6 - protein, 13 aa, Artificial Sequence
  Synthesized peptide P6
LDSPSEGRAPGAD

(19) United States
(12) Patent Application Publication
(10) Pub. No.: US 2023/0173014 A1
(43) Pub. Date: Jun. 8, 2023

(54) WINE LEES, DERIVATIVES THEREOF AND THEIR USES

(71) Applicant: GRANDES VINOS Y VIÑEDOS SA, CARIÑENA (Zaragoza) (ES)

(72) Inventors: Francisca Isabel BRAVO VÁZQUEZ, SANTA MARTA DE LOS BARROS (ES); Raúl LÓPEZ FERNÁNDEZ, BOLAÑOS DE CALATRAVA (ES); Juan María ALCAIDE HIDALGO, GRANADA (ES); María MARGALEF JORNET, Tarragona (ES); Anna MAS CAPDEVILA, REUS (ES); Jose María DEL BAS PRIOR, TARRAGONA (ES); María Eugenia HERNÁNDEZ DE PABLO, VALENCIA (ES); María Begoñia MUGUERZA MARQUÍNEZ, VALENCIA (ES)

(21) Appl. No.: 17/997,501

(22) PCT Filed: Feb. 9, 2021
(86) PCT No.: PCT/EP2021/053051

§ 371 Date: Oct. 28, 2022

(51) Int. Cl. A61K 38/01; C12N 9/54; A23L 33/105; A23L 33/18; A61P 9/12

(52) U.S. Cl. CPC A61K 38/01; C12N 9/54; A23L 33/105; A23L 33/18; A61P 9/12

(57) ABSTRACT

The present invention relates to wine lees and derivatives thereof. Likewise, it relates to their use in medicine.

### TECHNICAL FIELD

The present invention belongs to the field of cardiovascular diseases, particularly hypertension and diseases associated thereto. Specifically, it relates to peptides, wine lees and hydrolysates thereof, with antihypertensive activity.

### BACKGROUND OF THE INVENTION

The term antihypertensive means any substance or procedure that reduces blood pressure (BP). Several effective antihypertensive agents are known that are classified according to their mechanism of action. Thus, diuretics, beta adrenergic blockers, calcium channel blockers, central and peripheral adrenergic inhibitors, angiotensin receptor inhibitors or Angiotensin Converting Enzyme (ACE) inhibitors are identified. ACE catalyzes the conversion of angiotensin (Ang) I into Ang II, which is a vasoconstrictor hormone.

ACE inhibitors (ACE inhibitors) interfere with the production of Ang II by blocking the enzyme that produces it. Such an effect not only reduces BP but also decreases the vascular damage caused by hypertension, which in turn decreases the incidence of complications in the patient, particularly kidney failure or heart failure. Most of the peptides derived from dietary proteins with antihypertensive activity act by inhibiting ACE (ACE inhibitory activity, ACEI), since it is the target that is normally sought to inhibit, due to the great effectiveness shown by ACE inhibitor drugs in lowering BP in hypertensive people. In fact, they are the first treatments to use when diagnosing hypertension.

A number of peptides with ACEI activity have been described in the art, as for example in U.S. Pat. No. 8,673,862 B1. Various protein product hydrolysates comprising peptides with ACEI activity have also been described. For example, WO 2007004876 A2 describes the sequence of various peptides with ACEI activity in vitro obtained from a hydrolysate of milk proteins. WO 2007108554 A1 describes the obtaining from chicken collagen of an enzymatic hydrolysate with ACEI activity that also shows antihypertensive properties.

Not all peptides with ACEI activity (in vitro) show antihypertensive activity after oral ingestion. This is due to the action of the digestive enzymes on the peptides, which break them down and generate inactive forms or due to their inability to be absorbed at the intestinal level. Even peptides that do not have great ACEI activity in vitro, can acquire it in vivo due to the digestive process where active forms are generated. For this reason, it is essential to test the activity of the peptides in an in vivo model. Wine lees are the residue that is formed at the bottom of containers containing wine, after fermentation, during storage or after authorized treatments. Wine lees are mainly composed of microorganisms (mainly yeasts) and, to a lesser extent, tartaric acid and inorganic matter.

Currently the wine industry generates tons of lees. The usual destination of these wine lees is dealcoholization to obtain alcohol and the waste is destined for animal feed or as agricultural fertilizer, with little added value, or its destruction, which is an expense for the winery.

Thus, there is a need to revalue these abundant by-products of the wine industry. Surprisingly, the authors of the present invention have developed a new application of wine lees and their by-products in the field of medicine.

### OBJECT OF THE INVENTION

In a first aspect, the present invention relates to a wine lees hydrolysate or to said wine lees, for use in medicine.

A second aspect of the invention relates to a wine lees hydrolysate or to said wine lees, for use in the treatment and/or prevention of hypertension, a cardiovascular disease (CVD) associated with hypertension or a disease treated with ACE inhibitors, or for use as a coadjuvant in the treatment and/or prevention of hypertension, an CVD associated with hypertension or a disease treated with ACE inhibitors.

A third aspect, the present invention relates to a peptide for use in medicine, the sequence of which is selected from the group consisting of SEQ ID No. 1-SEQ ID No. 6.

A fourth aspect of the invention relates to a peptide for use in the treatment and/or prevention of hypertension, a CVD associated with hypertension or a disease treated with ACE inhibitors, or for use as a coadjuvant in the treatment and/or prevention of hypertension, a CVD associated with hypertension or a disease treated with ACE inhibitors, the sequence of which is selected from the group consisting of SEQ ID No. 1-SEQ ID No. 6.

A fifth aspect of the invention relates to a wine lees hydrolysate.

A sixth aspect of the invention relates to wine lees consisting of the supernatant from said lees.

A seventh aspect of the invention relates to a peptide the sequence of which is selected from the group consisting of SEQ ID No. 1-SEQ ID No. 6.

In an eighth aspect, the invention relates to a composition comprising the hydrolysate according to the fifth aspect of the invention, the lees according to the sixth aspect of the invention, the peptide according to the seventh aspect of the invention, and one or more vehicles, diluents, solvents, excipients, or additives.

A ninth aspect of the invention relates to a medicament, food supplement, beverage or food product characterized in that it comprises the hydrolysate of the fifth aspect of the invention, the lees of the sixth aspect of the invention, the peptide of the seventh aspect of the invention, or the composition of the eighth aspect of the invention.

A tenth aspect of the present invention relates to a method for preparing the hydrolysate of the fifth aspect of the invention.

An eleventh aspect of the invention relates to a hydrolysate obtainable by the method according to the tenth aspect of the invention and its uses.

Other objects, features, advantages and aspects of the present application will be apparent to the person skilled in the art from the description and the attached claims.

### BRIEF DESCRIPTION OF FIGURES

FIG. 1.—Graph showing the decrease in systolic blood pressure (ΔSBP; A) and diastolic blood pressure (ΔDBP; B) observed in spontaneously hypertensive rats (SHR) that are administered 1.5 mL of water (▪), 5 mL/kg of body weight (bw) of wine lees made with grapes of the Cabernet variety (⋄), 5 mL/kg bw of wine lees made with grapes of the Garnacha variety (∘), 5 mL/kg bw of wine lees made with grapes of the Mazuela variety (x), or 50 mg/kg bw of captopril (▴), depending on the time elapsed after administration.

FIG. 2.—Graph showing the ΔSBP (A) and ΔDBP (B) observed in spontaneously hypertensive rats (SHR) that are administered 1.5 mL of water (▪), 2.5 mL/kg of body weight (bw) of UVC wine lees (o), 5 mL/kg bw of UVC wine lees (⋄), 7.5 mL/kg bw of UVC wine lees (□) or 50 mg/kg bw of captopril (▴), depending on the time elapsed after the administration.

FIG. 3.—Graph showing the ΔSBP (A) and ΔDBP (B) observed in Wistar-kyoto rats that are administered 1.5 mL of water (▪) or 5 mL/kg bw of UVC wine lees (⋄) depending on the time elapsed after the administration.

FIG. 4.—Graphs showing ΔSBP (A) and ΔDBP (B) obtained in spontaneously hypertensive rats (SHR) that are administered 1.5 mL of water (▪), 5 mL/kg of body weight (bw) of UVC wine lees (0), 5 mL/kg bw of UVC wine lees hydrolysate (o), or 50 mg/kg bw of captopril (▴), depending on the time elapsed after administration.

FIG. 5.—Semi-preparative scale reverse phase HPLC chromatogram of the active supernatant obtained after centrifugation and ultrafiltration, through a 3,000 Da pore size membrane, of “hydrolysate 3”. F.1-F.9 correspond to the nine fractions collected.

### DETAILED DESCRIPTION OF THE INVENTION

As used in the present application, the singular forms “a/an”, “one” and “the” include their corresponding plurals unless the context clearly indicates otherwise. Unless otherwise defined, all technical terms used herein have the meaning that a person ordinary skilled in the art to which this invention pertain usually understands.

In order to facilitate understanding and clarify the meaning of certain terms in the context of the present invention, the following definitions and particular and preferred embodiments thereof are provided, applicable to all embodiments of the different aspects of the present invention.

In the context of the present invention, wine lees are the residue that is formed at the bottom of containers containing wine, after fermentation, during storage or after authorized treatments. Likewise, it is considered as lees the solid phase obtained after filtration or centrifugation of the residue that is formed at the bottom of containers containing wine, after fermentation, during storage or after authorized treatments, and the liquid phase (also referred to hereafter as supernatant) obtained with solubilization, extraction and/or separation processes of the residue that is formed at the bottom of containers containing wine, after fermentation, during storage or after authorized treatments. Among these separation processes are, among others, filtration, centrifugation, sedimentation and dialysis. In a preferred embodiment, the liquid phase is obtained by centrifugation or filtration.

In the present invention, the wine relates to the product obtained by alcoholic fermentation, total or partial, of fresh grapes, crushed or not, or of grape must. Thus, it does not relate to alcoholic beverages from the fermentation of other products, such as, for example, rice (sake).

As used herein, the term “prevent” or “prevention” means preventing, reducing the risk, or delaying the onset of symptoms of a medical condition or disease.

“Prophylactically effective amount” refers to an amount that, when administered, prevents, reduces the risk, or delays the onset of symptoms of a medical condition or disease. “Treatment” relates to the reduction or elimination of symptoms of a medical condition or disease.

“Therapeutically effective amount” refers to an amount that, when administered, reduces or eliminates symptoms of a medical condition or disease.

As defined by the World Health Organization, hypertension, also known as high or elevated blood pressure, is a disorder in which blood vessels have persistently high blood pressure, which can damage them.

By “cardiovascular disease (CVD) associated with hypertension” is understood that CVD that is due, in part or in its entirety, to the presence of hypertension or that is worsened by the presence of hypertension. In a particular embodiment, CVD is selected from the group consisting of coronary heart disease, cerebrovascular disease, heart failure, peripheral vascular disease, rheumatic heart disease, congenital heart disease, cardiomyopathies.

By “ACE inhibitor-treated disease” is meant a disease whose symptoms can be or are prevented, treated, or ameliorated with the administration of ACE inhibitors. In a particular embodiment, the disease treated with ACE inhibitors is selected from the group consisting of heart failure, coronary artery disease, diabetes, and chronic kidney disease.

The authors of the present invention have characterized different wine lees with ACEI activity in vitro and/or antihypertensive activity in vivo. Thus, said lees and their by-products (e.g. supernatant, hydrolysate, peptides) have a medical use, particularly for the treatment and/or prevention of hypertension.

Thus, in a first aspect, the present invention relates to wine lees for use in medicine. Likewise, it relates to a wine lees hydrolysate for use in medicine.

Likewise, the first aspect of the invention relates to the use of wine lees or wine lees hydrolysate for preparing a medicament.

A second aspect of the invention relates to wine lees for use in the treatment and/or prevention of hypertension, a CVD associated with hypertension or a disease treated with ACE inhibitors. Likewise, it relates to wine lees for use as coadjuvant in the treatment and/or prevention of hypertension, a CVD associated with hypertension or a disease treated with ACE inhibitors.

The second aspect of the invention also relates to a wine lees hydrolysate for use in the treatment and/or prevention of hypertension, a CVD associated with hypertension or a disease treated with ACE inhibitors. Likewise, it relates to a wine lees hydrolysate for use as coadjuvant in the treatment and/or prevention of hypertension, a CVD associated with hypertension or a disease treated with ACE inhibitors.

Likewise, the second aspect of the invention relates to the use of wine lees or a wine lees hydrolysate for preparing a medicament or a coadjuvant for the treatment and/or prevention of hypertension, a CVD associated with hypertension or to ACE.

The second aspect of the invention also relates to a method of treating hypertension, a CVD associated with hypertension or a disease treated with ACE inhibitors in a subject (mammal, preferably human), comprising administering, preferably a therapeutically effective amount, to said subject of wine lees or a wine lees hydrolysate. The second aspect of the invention also relates to a method of preventing hypertension, a CVD associated with hypertension or a disease treated with ACE inhibitors in a subject (mammal, preferably human), comprising administering, preferably a prophylactically effective amount, to said subject of wine lees or a wine lees hydrolysate. In a preferred embodiment according to any one of the preceding embodiments, administration is carried out orally. The dosage will depend on various factors such as the subject's weight, sex, type and severity of the conditions to be treated, etc. and it will be easily determined by the skilled in the art.

As shown in the examples, wine lees made from Cabernet grapes (hereafter referred to as “UVC wine lees”) and Tempranillo grapes (hereafter referred to as “UVT wine lees”) have the highest in vitro ACEI activities (Table 1) among the different lees analyzed. Specifically, UVC wine and UVT wine lees have an ACEI activity with an IC₅₀ de 0.15 μL y 0.08 μL, respectively. On the other hand, hydrolysates have ACEI activity with an IC₅₀ de 0.19 μL, 0.18 μL y 0.16 μL (Table 5).

Thus, in a particular embodiment according to any one of the embodiments of the first and second aspect of the invention, the wine lees are wine lees from grapes of the Cabernet variety and/or Tempranillo variety, more preferably from grapes of the Cabernet variety.

In another particular embodiment according to any one of the embodiments of the first and second aspect of the invention, the wine lees and their hydrolysate have an ACEI activity in vitro with an IC₅₀ lower than or equal to 0.25 μL, preferably lower than or equal to 0.20 μL.

More particularly, the wine lees are wine lees from grapes of the Cabernet variety and/or Tempranillo variety and the wine lees and their hydrolysate have an ACEI activity in vitro with an IC₅₀ lower than or equal to 0.25, preferably lower than or equal to 0.20 μL.

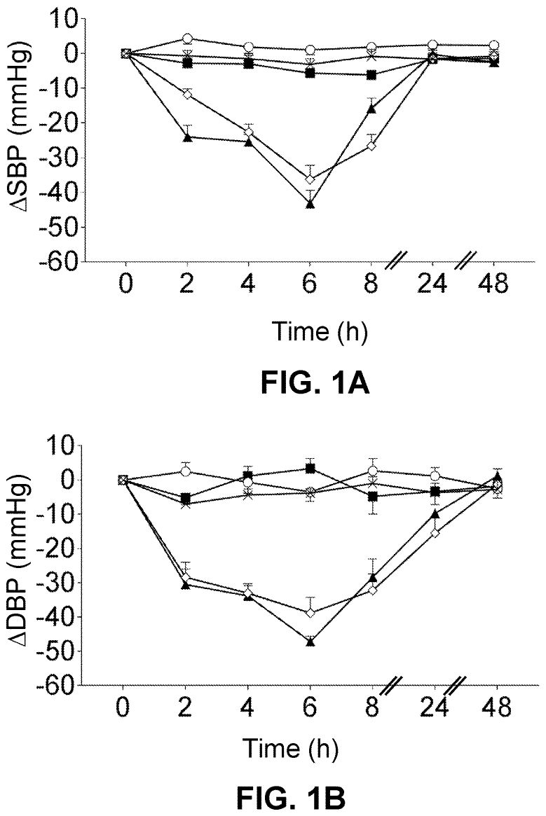

As shown in the examples, the UVC wine lees were analyzed in more detail and showed antihypertensive activity in vivo (FIG. 1, Example 3). This, however, does not happen with wine lees made with grapes of the Garnacha or Mazuela variety (FIG. 1). Since the IC₅₀ of UVT wine lees is lower than that of UVC wine lees, it is expected that these lees also have antihypertensive activity in vivo. Thus, in a preferred embodiment according to any one of the embodiments of the first and second aspect of the invention, the lees have antihypertensive activity in vivo. More preferably, the wine lees are UVC wine lees and/or UVT wine lees, more preferably they are UVC wine lees.

Interestingly, it should be noted that UVC wine lees from different wineries showed the same ACEI activity in vitro. This suggests that the ACEI activity is independent of the source winery (Example 1).

As shown in Example 4, advantageously, drying of the UVC wine lees did not reduce the antihypertensive effectiveness shown by them. In fact, it increased its effectiveness from the two hours after their administration, and their effect was more prolonged in time. Thus, in a particular embodiment according to any one of the embodiments of the first and second aspect of the invention, the wine lees or the hydrolysate are dry, in a solid state. Also, as dry wine lees, which do not have alcohol, have a greater antihypertensive effect, in another particular embodiment according to any one of the embodiments of the first and second aspect of the invention, the wine lees are dealcoholized (have no alcohol or have an alcohol content lower than or equal to 0.01%).

Interestingly, the results of Example 6 allow to rule out possible undesirable effects of the UVC wine lees on the BP of normotensive subjects and indicate that the lees only exert their antihypertensive action when there is a pathological state of hypertension. Thus, it is safe to administer the lees of the invention to prevent hypertension in normotensive patients.

In a particular embodiment according to any one of the embodiments of the first and second aspect of the invention, wine lees supernatant has ACEI activity in vitro with an IC₅₀ lower than or equal to 0.25 μL, preferably lower than or equal to 0.20 μL, and/or antihypertensive activity in vivo. More particularly, the wine lees consist of the supernatant from said lees, which has ACEI activity in vitro with an IC₅₀ lower than or equal to 0.25 μL, preferably lower than or equal to 0.20 μL, and/or antihypertensive activity in vivo. More particularly, the supernatant is obtained by centrifugation and/or filtration, and more preferably it is obtained by centrifugation.

In another particular embodiment according to any one of the embodiments of the first or second aspect of the invention, said supernatant comprises at least 60% proline, preferably at least 65% proline, and optionally comprises less than 4% of each of asparagine, glutamine and valine. Preferably, the wine lees are from grapes of Cabernet variety and/or Tempranillo variety and consisting of the supernatant from said lees, preferably said supernatant comprises at least 60% proline, and optionally comprises less than 4% of each of asparagine, glutamine and valine. More preferably, the content of said amino acids is determined following the method described by Mas-Capdevila et al. 2019 (“Long-term administration of protein hydrolysate from chicken feet induces antihypertensive effect and confers vasoprotective pattern in diet-induced hypertensive rats” 2019, Journal of Functional Foods, 55, 28-35). This method was used to characterize the amino acid content of the hydrolysates and wine lees of the examples (data not shown).

As shown in Example 8, the wine lees enzyme hydrolysate has antihypertensive activity in vivo. Thus, in a preferred embodiment of any one of the embodiments of the first and second aspect of the invention, the wine lees hydrolysate has antihypertensive activity in vivo.

As shown in Example 9, said hydrolysate comprises a series of peptides with ACEI activity in vitro. Thus, in a particular embodiment according to any one of the embodiments of the first and second aspect of the invention, the hydrolysate comprises a peptide of sequence selected from the group formed by SEQ ID No. 1-SEQ ID No. 6 and combinations thereof, that is it comprises one, several (two, three, four or five) or all the peptides of sequence selected from the group formed by SEQ ID No. 1-SEQ ID No. 6.

As shown in Example 9, the peptides of sequence SEQ ID No. 1 and 6 are those with the highest antihypertensive activity in vivo. Thus, in a particular embodiment according to any one of the embodiments of the first and second aspect of the invention, the hydrolysate comprises a peptide of sequence SEQ ID No. 1 and/or a peptide of sequence SEQ ID No. 6. More particularly, the hydrolysate comprises two, three, four, five or six peptides of a sequence selected from the group consisting of SEQ ID No. 1-SEQ ID No. 6, at least one of them being the peptide of sequence SEQ ID No. 1 or SEQ ID No. 6.

It is considered that small reductions in BP significantly reduces the risk of all major cardiovascular outcomes and all-cause death in patients with hypertension. In this sense, it has been observed that a reduction in 10 mmHg for SBP and 5 mmHg for DBP is accompanied by a 20% reduction in coronary heart disease events, 36% reduction in stroke, 27% reduction in major cardiovascular events, 38% reduction in hospitalized heart failure and 16% reduction in cardiovascular death (Thomopoulos C, Parati G, Zanchetti A. “Effects of blood pressure lowering on outcome incidence in hypertension. 1. Overview, meta-analyses and meta-regression analyses of randomized trials”. Journal of Hypertension 2014; 32:2285-2295). As shown in the examples, all the products of the present invention (e.g. wine lees, wine lees hydrolysates and peptides) are capable of reducing the SBP by 10 mmHg or more and the DBP by at least 5 mmHg, so they are useful for the treatment and/or prevention of hypertension itself and of CVD associated with hypertension. Likewise, since they have high ACEI activity, they are useful for the treatment and/or prevention of diseases treated with ACE inhibitors.

The uses and methods described in the first and second aspects of the invention represent a great advantage over chemical treatments since wine lees and their enzymatic hydrolysates are ingredients of natural origin that are non-toxic or have minimal toxicity.

The particular and preferred embodiments described in the first and second aspects of the invention regarding lees, hydrolysates and their peptides are applicable to the other aspects of the present invention.

Thanks to their antihypertensive activity, the peptides of the hydrolysates of the invention can be used in medicine, particularly for the treatment and/or prevention of hypertension. Thus, in a third aspect, the present invention relates to a peptide for use in medicine, the sequence of which is selected from the group consisting of SEQ ID No. 1-SEQ ID No. 6, more particularly it relates to a peptide the sequence of which is SEQ ID No. 1 or SEQ ID No. 6. Likewise, the third aspect of the invention relates to the use of a peptide for preparing a medicament, the sequence of which is selected from the group consisting of SEQ ID No. 1-SEQ ID No. 6, more particularly it relates to an isolated peptide the sequence of which is SEQ ID No. 1 or SEQ ID No. 6. In a particular embodiment according to any one of the preceding embodiments, the peptide has antihypertensive activity in vivo. In a particular embodiment according to any one of the preceding embodiments, the peptide is an isolated peptide.

Likewise, a fourth aspect of the invention relates to a peptide for use in the treatment and/or prevention of hypertension, a CVD associated with hypertension or a disease treated with ACE inhibitors, or for use as a coadjuvant in the treatment and/or prevention of hypertension, a CVD associated with hypertension or a disease treated with ACE inhibitors, the sequence of which is selected from the group consisting of SEQ ID No. 1-SEQ ID No. 6, more particularly it relates to an isolated peptide the sequence of which is SEQ ID No. 1 or SEQ ID No. 6.

The fourth aspect of the invention also relates to the use of a peptide for preparing a medicament or a coadjuvant for the treatment and/or prevention of hypertension, a CVD associated with hypertension or a disease treated with ACE inhibitors, the sequence of which is selected from the group consisting of SEQ ID No. 1-SEQ ID No. 6, more particularly the sequence of which is SEQ ID No. 1 or SEQ ID No. 6.

The fourth aspect of the invention also relates to a method of treating hypertension, a CVD associated with hypertension or a disease treated with ACE inhibitors in a subject (mammal, preferably human), comprising administering, preferably a therapeutically effective amount, to said subject of a peptide as defined in any one of the embodiments of the fourth aspect of the invention. Likewise, the fourth aspect of the invention also relates to a method of preventing hypertension, a CVD associated with hypertension or a disease treated with ACE inhibitors in a subject (mammal, preferably human), comprising administering, preferably a prophylactically effective amount, to said subject of a peptide as defined in any one of the embodiments of the fourth aspect of the invention. In a preferred embodiment according to any one of the preceding embodiments, administration is carried out orally.

In a particular embodiment according to any one of the embodiments of the fourth aspect of the invention, the peptide has antihypertensive activity in vivo.

In another particular embodiment according to any one of the embodiments of the fourth aspect of the invention, the peptide is an isolated peptide.

As mentioned above, the authors of the present invention have developed a wine lees enzyme hydrolysate having antihypertensive activity. Thus, in a fifth aspect, the present invention relates to a hydrolysate as defined in any one of the embodiments of the first and second aspect of the invention. For example, in a particular embodiment according to any one of the embodiments of the fifth aspect of the invention, the hydrolysate comprises one, several (two, three, four or five) or all the peptides of the sequence selected from the group consisting of SEQ ID No. 1-SEQ ID No. 6, more preferably it comprises a peptide of sequence SEQ ID No. 1 and/or a peptide of sequence SEQ ID No. 6. In another particular embodiment according to any one of the embodiments of the fifth aspect of the invention, the lees are UVC wine and/or UVT wine lees, more preferably UVC wine lees. In another particular embodiment according to any one of the embodiments of the fifth aspect of the invention, the hydrolysate has ACEI activity in vitro with an IC₅₀ lower than or equal to 0.25 μL, preferably lower than or equal to 0.20 μL, and/or antihypertensive activity in vivo.

The fifth aspect of the invention also refers to a wine lees hydrolysate, wherein the wine lees are wine lees of grapes of the Cabernet variety and/or Tempranillo variety. More particularly, said hydrolysate has ACEI activity in vitro with an IC₅₀ lower than or equal to 0.25 μL, preferably lower than or equal to 0.20 μL, and/or antihypertensive activity in vivo.

As mentioned above, the authors of the present invention have determined that the supernatant of the wine lees has ACEI and/or antihypertensive activity. Thus, in a sixth aspect, the present invention relates to wine lees consisting of the supernatant from said lees and having ACEI activity in vitro with an IC₅₀ lower than or equal to 0.25 μL, preferably lower than or equal to 0.20 μL, and/or antihypertensive activity in vivo. More particularly, said supernatant comprises at least 60% proline, preferably at least 65% proline, and optionally comprises less than 4% of each of asparagine, glutamine, and valine. In another particular embodiment, according to any one of the preceding embodiments, the lees are UVC wine and/or UVT wine lees.

Preferably, the sixth aspect of the invention refers to wine lees from grapes of Cabernet variety and/or Tempranillo variety and consisting of the supernatant from said lees, preferably said supernatant comprises at least 60% proline, and optionally comprises less than 4% of each of asparagine, glutamine and valine. More preferably, said wine lees have ACEI activity in vitro with an IC₅₀ lower than or equal to 0.25 μL, preferably lower than or equal to 0.20 μL, and/or antihypertensive activity in vivo. As mentioned above, the authors of the present invention have isolated peptides that have antihypertensive activity in vivo. Thus, in a seventh aspect, the present invention relates to an isolated peptide the sequence of which is selected from the group consisting of SEQ ID No. 1-SEQ ID No. 6, more particularly it relates to an isolated peptide the sequence of which is SEQ ID No. 1 or SEQ ID No. 6.

This aspect also refers to a nucleic acid, encoding a peptide according to the seventh aspect of the invention, optionally linked to a heterologous promoter sequence. Likewise, it refers to an expression vector capable of expressing or expressing said nucleic acid, and to a host cell, preferably recombinant host cell, comprising the peptide, the nucleic acid, or the expression vector of the seventh aspect of the invention.

The host cell can be either prokaryotic or eukaryotic. Bacterial cells may be preferred prokaryotic host cells. Preferred eukaryotic host cells include yeast, insect and mammalian cells, preferably vertebrate cells such as those from a mouse, rat, monkey, or human fibroblastic and colon cell lines. More preferably, the host cell is a yeast, e.g. Saccharomyces cerevisiae.

The methods for transformation of the cell hosts are well-known by the skilled in the art.

The seventh aspect of the invention also refers to the nucleic acid, the vector or the host cell as defined above for use in medicine, in particular for use in the treatment and/or prevention of hypertension, a CVD associated with hypertension or a disease treated with ACE inhibitors, or for use as coadjuvant in the treatment and/or prevention of hypertension, a CVD associated with hypertension or a disease treated with ACE inhibitors.

The hydrolysate, lees, peptides, nucleic acid, vector, and host cell can be formulated into different compositions. Thus, in an eighth aspect, the invention relates to a composition comprising the hydrolysate according to any one of the embodiments of the fifth aspect of the invention, and/or the wine lees as defined in any one of the embodiments of the first and sixth aspect of the invention, and/or the isolated peptide, nucleic acid, vector or host cell according to any one of the embodiments of the seventh aspect of the invention, and one or more vehicles, diluents, solvents, excipients, or additives. In a particular embodiment according to any one of the preceding embodiments, the composition is a nutraceutical, pharmaceutical, or food composition.

The vehicles, diluents, solvents, excipients, or additives are widely known to the person skilled in the art, who knows which one to choose according to the type of composition and the desired administration form. For example, pharmaceutically acceptable excipients may be as described in “Remington's Pharmaceutical Sciences Handbook” Mack Pub. Co., N.Y. USA The hydrolysate, lees, peptides, nucleic acid, vector, host cell and composition defined above can be formulated as medicament, food supplement, beverage, or food product. Thus, in a ninth aspect, the invention relates to a medicament, a food supplement, a beverage or a food product characterized in that it comprises the hydrolysate according to any one of the embodiments of the fifth aspect of the invention, and/or the wine lees as defined in any one of the embodiments of the first and sixth aspect of the invention, and/or the isolated peptide, nucleic acid, vector or host cell according to any one of the embodiments of the seventh aspect of the invention, and/or the composition according to any one of the embodiments of the eighth aspect.

To be formulated as a medicament or food supplement, the hydrolysate, lees, peptides, nucleic acid, vector, host cell or compositions of the invention, can be combined with any vehicle, diluent, adjuvant, excipient, etc. suitable to obtain the medicament or supplement in the desired administration form. Thus, they can be presented in any administration form, solid or liquid, and administered by any appropriate route, oral, respiratory, rectal, or topical. Advantageously, the medicament or food supplement is administered orally. Examples of such formulations, which can be prepared using well known methods and excipients, such as those described in, “Remington's Pharmaceutical Sciences Handbook” Mack Pub. Co., N.Y. USA, are tablets, capsules, syrups, and the like for oral administration, while for parental administration the suitable forms are sterile solutions or suspensions in acceptable liquids, implants, etc.

To be formulated as a beverage or food product, the hydrolysate, lees, peptides, nucleic acid, vector, host cell or compositions of the invention can be combined with any common food ingredient. The term “beverage” is intended to include liquids and syrups, as well as powder formulations to be dissolved in water or another liquid component for the preparation of instant beverages. In a particular embodiment according to any one of the embodiments of the ninth aspect of the invention, the beverage is water, a milk beverage, an energy beverage, a soft beverage, a juice, or wine, preferably the beverage is wine, so any wine, e.g. any red, white, rosé, sparkling wine can comprise the hydrolysate, the lees, the peptides or the compositions of the invention.

A tenth aspect of the invention relates to a method for obtaining a hydrolysate as defined in any one of the embodiments of the fifth aspect of the invention. Particularly, the method comprises the following steps:
a) Enzymatic hydrolysis of wine lees with one or more enzymes selected from the group consisting of protease, serine protease or aminopeptidase, under conditions suitable for the selected enzyme/s;
b) Inactivation of the enzyme.

In a particular embodiment of the tenth aspect of the invention, the wine lees are from grapes of Cabernet variety and/or Tempranillo variety, preferably Cabernet variety.

In another particular embodiment of the tenth aspect of the invention, step a) is carried out with one or more enzymes selected from the group consisting of:
protease from Bacillus amyloliquefaciens, preferably E.C. 3.4.24.28,
serine protease from Bacillus licheniformis, preferably E.C. 3.4.21.62, and
aminopeptidase from Aspergillus oyzae, preferably E.C. 3.4.11.1.

More particularly, the enzyme is a combination of the protease and serine protease mentioned above. Preferably, the enzyme is an aminopeptidase from Aspergillus oyzae, more preferably E.C. 3.4.11.1.

As shown in the examples, the hydrolysates obtained with said enzymes have an ACEI activity with an IC₅₀ lower than 0.20 μL and the hydrolysate obtained with the aminopeptidase has excellent antihypertensive activity in vivo.

As for the hydrolysis conditions, the person skilled in the art knows how to choose the conditions for each enzyme. In a particular embodiment of the invention, aminopeptidase of A. oyzae, preferably E.C. 3.4.11.1, is used at pH from 3.5 to 7.5, more particularly from 4 to 5,5, and preferably from 4 to 5. In another particular embodiment, the protease of Bacillus amyloliquefaciens, preferably E.C. 3.4.24.28, and the serine protease of Bacillus licheniformis, preferably E.C. 3.4.21.62, are used at a pH from 6.0 to 8.5, more particularly from 6.5 to 7.5. Regarding temperature, in a particular embodiment according to any one of the preceding embodiments, the hydrolysis temperature is from 20° C. to 55° C., more particularly from 25° C. to 50° C., and preferably from 25° C. to 40° C. Regarding time, in a particular embodiment according to any one of the preceding embodiments, the hydrolysis is carried out from 1 h to 24 h, preferably from 1.5 h to 4 h, and more preferably from 1.5 h to 2.5 h, or for 2 h. In a preferred embodiment according to any one of the preceding embodiments, the hydrolysis is carried out at a temperature of 25° C. and at pH 4 or 7, depending on the enzyme as shown in the examples.

As for wine lees, in a particular embodiment, the wine lees have ACEI activity in vitro with an IC₅₀ lower than or equal to 0.25 μL and/or antihypertensive activity in vivo. As for step b), the inactivation of the enzyme can be carried out by any method known by the person skilled in the art, such as, for example, by lowering the pH, heating, or centrifuging. In a particular embodiment according to any one of the preceding embodiments, the inactivation will be carried out by lowering the pH or by heating. More particularly, when the enzyme is an aminopeptidase from Aspergillus oyzae, preferably E.C. 3.4.11.1, inactivation is carried out by lowering the pH, and when the enzyme is a protease from Bacillus amyloliquefaciens, preferably E.C. 3.4.24.28, and/or a serine protease from Bacillus licheniformis, preferably E.C. 3.4.21.62, inactivation is carried out by heating, particularly at least 80° C. for at least 10 minutes. In another particular embodiment according to any one of the embodiments of the tenth aspect of the invention, the method further comprises the following steps:

c) Evaluation of the ACEI activity; and

d) Selection of a hydrolysate with an ACEI activity in vitro with an IC₅₀ lower than or equal to 0.25 μL.

Regarding step c), in a particular embodiment according to any one of the preceding embodiments, the method for evaluating or measuring ACEI activity is the method described by Mas-Capdevila et al. 2018 (“Dose-related antihypertensive properties and the corresponding mechanisms of a chicken foot hydrolysate in hypertensive rats”. Nutrients. 2018, 10(9), 1295).

Regarding step d), in a particular embodiment according to any one of the preceding embodiments, a hydrolysate having an ACEI activity in vitro with an IC₅₀ lower than or equal to 0.20 μL is chosen.

Finally, in an eleventh aspect, the invention relates to a hydrolysate obtainable by the method according to any one of the embodiments of the tenth aspect of the invention. Likewise, it relates to said hydrolysate for use in medicine and for use in the treatment and/or prevention of hypertension, a CVD associated with hypertension or a disease treated with ACE inhibitors, or for use as a coadjuvant in the treatment and/or prevention of hypertension, a CVD associated with hypertension or a disease treated with ACE inhibitors. It also relates to a composition comprising said hydrolysate, and one or more vehicles, diluents, solvents, excipients, or additives, preferably the composition is a nutraceutical, pharmaceutical, or food composition. Likewise, it relates to a medicament, a food supplement, a beverage, or a food product comprising said hydrolysate. Finally, it also relates to a method of treating or preventing hypertension, a CVD associated with hypertension or a disease treated with ACE inhibitors in a subject (mammal, preferably human), comprising administering, preferably a therapeutically or prophylactically effective amount, respectively, to said subject of the nucleic acid, vector or host cells defined above. The particular embodiments of the method, hydrolysate, the compositions, medicaments, etc., of the second, fifth, eighth and ninth aspects, respectively, are applicable to the eleventh aspect of the present invention.

### EXAMPLES

With the intention of showing the present invention in an illustrative way, but not in a limiting way, the following examples are provided. All the procedures carried out on the animals in the following examples were approved by the Ethical Committee of the Universitat Rovira i Virgili.

### Example 1. Wine Lees with Antihypertensive Activity

Wine lees from different grape varieties were collected at the time of the wine transfer. The grape varieties from which the wine was made were Garnacha, Tempranillo, Cabernet, Merlot and Mazuela as representatives of red grapes and Macabeo of white grapes.

Once the wine lees were collected, they were centrifuged at 2,950×g, 15 min, 4° C. and the supernatant was collected. The ACEI activity of the supernatants was determined as described in Example 2.

Table 1 shows the results obtained from the aforementioned determination represented as a percentage of IC₅₀ (4). The IC₅₀ represents the volume of the hydrolysate (4) necessary to inhibit 50% of the ACE enzyme, the lower the value of the enzyme, the greater the inhibitory activity of the wine lees.

TABLE 1
ACEI activity of wine lees from different grape varieties.

| Type of wine lees | ACEI activity | ACEI activity |
|---|---|---|
| (grape variety) | (%)* | (IC₅₀ in μL) |
| Tempranillo (red grape) | 71.81 ± 2.46 | 0.08 |
| Cabernet (red grape) | 55.7 ± 1.9 | 0.15 |
| Merlot (red grape) | 28.8 ± 0.3 | 0.33 |
| Garnacha (red grape) | 44.2 ± 2.5 | 0.44 |
| Mazuela (red grape) | 50.7 ± 8.1 | ND |
| Macabeo (white grape) | <9 | 3.71 |

*Dilution used 1/256;
ND: Undetermined

As shown in Table 1, the lees from the varieties used for making red wines showed greater ACEI potential than the lees from white varieties. Among the red varieties, those from the Cabernet and Tempranillo varieties showed the highest ACEI activity (lowest IC₅₀).

In order to check the repeatability of ACE inhibition by these lees, the lees from the Cabernet variety (UVC wine lees) from two different wineries were collected and their ACEI activity was determined. The results showed that both wine lees presented the same ACEI activity (IC₅₀=0.15 μL) regardless of its source.

### Example 2: Measurement of the ACEI Activity of the Wine Lees and Hydrolysates

The ACEI activity measurement of the wine lees and their hydrolysates was performed according to the method described by Mas-Capdevila et al. 2018 (“Dose-related antihypertensive properties and the corresponding mechanisms of a chicken foot hydrolysate in hypertensive rats”. Nutrients. 2018, 10 (9), 1295), after centrifugation at 2,950×g, 15 min, 4° C. and a dilution of 1/256.

The percentage of inhibition (% Inhibition) was calculated applying the following formula:

$$\% Inhibition = 1 - \frac{( Sample ) - ( Blank Sample )}{( Control ) - ( Blank Substrate )} \times 100$$

The IC₅₀ was calculated by determining the ACEI percentage of different concentrations of the sample. These data were graphically represented to obtain an equation of the line. Subsequently, the necessary amount of the sample was calculated to inhibit 50% of the enzyme by extrapolating in the previously obtained straight line. Subsequently, the necessary amount of the sample was calculated to inhibit 50% of the enzyme by extrapolating in the previously obtained straight line.

### Example 3: Antihypertensive Activity of the Wine Lees after Acute Administration in Hypertensive Rats

The present example describes the effect of different lees, UVC wine lees and lees obtained from winemaking with grapes of the Garnacha and Mazuela variety, on systolic and diastolic blood pressure (SBP and DBP, respectively) in spontaneously hypertensive rats (SHR). The effect on SBP of different doses of UVC wine lees is also described, also in SHR rats.

For the study, male SHR rats 17-20 weeks old and weighing between 310 and 360 g, from Charles River Laboratories España S.A. were used. The rats remained at a stable ambient temperature of 23° C., and with light-dark cycles of 12 hours, eating food and water ad-libitum.

The BP measurement in these animals was carried out with a modification of the tail-cuff technique, following the methodology and equipment described by Mas-Capdevila et al. 2018. The measurement of the SBP was performed prior to the administration of the treatments and after their administration at 2, 4, 6, 8, 24 and 48 h. The treatments consisted of the acute administration of a dose of 5 mL/kg bw of the different wine lees (supernatant collected after centrifugation at 2,950×g, 15 min, 4° C.). The effect of water administration (1.5 mL, negative control) was also evaluated in order to know the circadian variation of SBP in rats and of captopril (50 mg/kg bw dissolved in 1.5 mL of water), antihypertensive drug used as a positive control. The administration of all the treatments was carried out by means of an intragastric tube at between 9 and 10 a.m.

The SBP and DBP results are represented as a decrease in SBP or DBP at different times with respect to the SBP or DBP observed at 0 h (without treatment) for each animal (FIG. 1). Each data represents the mean±the ESM (n=6). Furthermore, the data of the different groups were compared in a 2-way analysis of variance (ANOVA) using the Tukey test at each post-administration time with the SPSS statistical program (IBM SPSS Statistics software version 20.0) and the difference was considered significant for p values <0.05.

FIGS. 1A and B show the decrease in SBP (ΔSBP) and in DBP (ΔDBP), respectively, observed in SHR rats at different times, after administration of the different wine lees, captopril and water. As expected, animals that received water did not show changes in their BP. In contrast, Captopril administration (50 mg/kg bw) led to a continuous decrease in the animals SBP and DBP 2 h post-treatment. The maximum decreases were observed at 6 h (−43.2±3.9 and −47.2±1.5 mmHg for SBP and DBP, respectively). Regarding the wine lees, only UVC wine lees showed an antihypertensive effect on both SBP and DBP in SHR, being their behavior similar to the one observed by Captopril. The maximum decrease in BP was also observed at 6 h post-administration (−36.4±3.4 and −38.8±4.6 mmHg for SBP and DBP, respectively). Initial BP values were recovered at 24 or 48 h for SBP and DBP, respectively. No significant changes in BP were found between the Garnacha or Mazuela WL groups and water group (FIGS. 1A and B). UVC wine lees were selected according their antihypertensive effect for further studies.

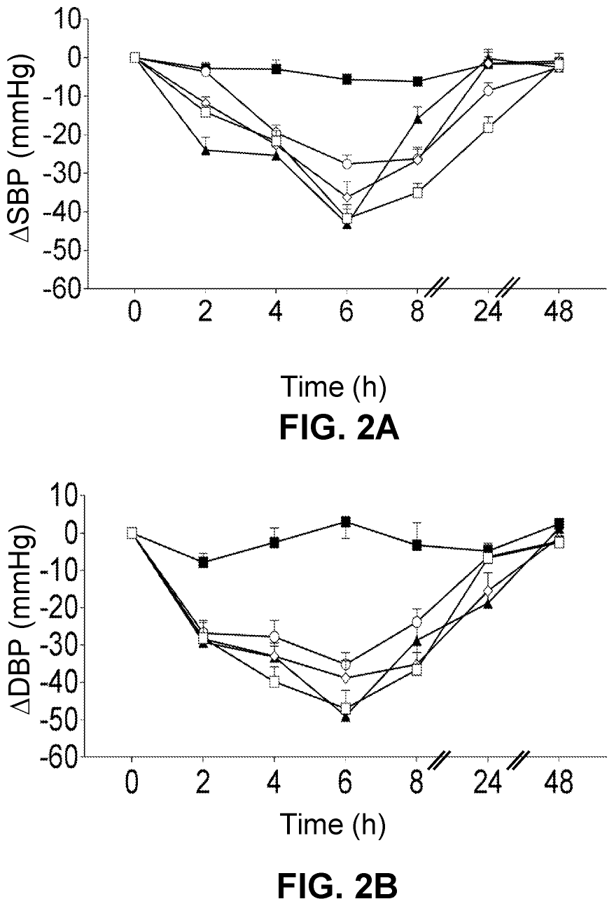

The antihypertensive effect of different doses of UVC wine lees was also tested (FIG. 2). As can be seen in FIG. 2A, the 3 doses tested (2.5, 5 and 7.5 mL/kg bw) produced a significant drop in SBP (compared to the SBP of the animals treated with water), showing their antihypertensive effects. More specifically, in all of them, the maximum decrease in SBP occurred at 6 h after administration (−27, −36 and −41 mmHg for doses of 2.5, 5 and 7.5 mL/kg bw, respectively). At that time, no significant differences were observed in the two highest doses with respect to the effect produced by captopril (−43 mmHg). Regarding DBP (FIG. 2B), all doses produced a significant reduction in DBP, being maximum at 6 h post administration (−35, −38 and −47 mmHg for doses of 2.5, 5 and 7.5 mL/kg bw, respectively). No significant differences were found between the different treatments and Captopril. Initial SBP and DBP in 5 and 7.5 mL doses values after UVC administration were not recovered until 48 h post-administration.

### Example 4. Antihypertensive Activity of Lyophilized UVC Wine Lees, after Acute Administration in Hypertensive Rats

In the present example, the effect of dry UVC wine lees, without alcohol, on its antihypertensive activity is described by means of its acute administration to SHR rats. The study was carried out following the method and conditions described in Example 3. The treatments that were evaluated consisted of the acute administration of water (1.5 mL), captopril (50 mg/kg bw), UVC wine lees (5 mL/kg bw), or the same lyophilized lees (125 mg/kg bw). The tested dose of lyophilized lees (125 mg/kg bw) corresponded to the same amount of dry product that is in 5 mL/kg bw of the wine lees. Solid state treatments were diluted in water for administration to animals. As can be seen in Table 2, drying of the UVC wine lees did not reduce the antihypertensive effectiveness shown by the lees. In fact, it increased its effectiveness from the two hours after its administration, producing a drop in systolic blood pressure of −33 mmHg compared to −12 mmHg, a drop produced by those without drying. Furthermore, its effect was more prolonged in time. Specifically, lyophilized UVC wine lees produced a drop in SBP of −41 mmHg at 24 h post-administration. These results show that alcohol-free lees have a greater antihypertensive effect.

TABLE 2
Decreased of systolic blood pressure (SBP) after oral administration
of the UVC wine lees to hypertensive rats.

| | ΔSBP (mmHg)* | | | | | |
|---|---|---|---|---|---|---|
| Treatment | 2 h | 4 h | 6 h | 8 h | 24 h | 48 h |
| Water | −4 ± 1 | −4 ± 2 | −7 ± 0 | −7 ± 1 | −3 ± 2 | −2 ± 1 |
| Captopril | −24 ± 3 | −26 ± 2 | −43 ± 4 | −16 ± 3 | −1 ± 2 | −2 ± 3 |
| UVC wine | −12 ± 2 | −22 ± 3 | −36 ± 4 | −26 ± 3 | −2 ± 3 | −1 ± 2 |
| lees | | | | | | |
| Lyophi- | −33 ± 2 | −44 ± 3 | −48 ± 5 | −37 ± 4 | −41 ± 3 | −2 ± 1 |
| lized UVC | | | | | | |
| wine lees | | | | | | |

*Decrease calculated as: SBP of the different hours − SBP of 0 h. Data represented as Mean ± ESM (n = 6).

### Example 5: Antihypertensive Activity of UVC Wine Lees after Chronic Administration in Hypertensive Rats

The present example describes the effect on SBP of hypertensive rats that were administered lyophilized UVC wine lees daily for 5 weeks.

Male SHR rats, 16 weeks old and weighing 307±19 g, from Charles River Laboratories Espana S.A. were used to assess the antihypertensive effect of lyophilized lees. Animal SBP was monitored by radiotelemetry using a Data Sciences International (DSI; St. Paul, Minn.) system consisting of the following components: MX2: 2.0, RPC-1 receptor for cages; HD-S10 sensor (catheter measurements: 8 cm×6 mm), APR-2 ambient pressure detector, data acquisition and analysis system Powerlab 16/35 and labChartPro. To use this system, it was necessary to operate the animals to insert the HD-S10 sensor into the abdominal aorta. The sensor was gently placed in the lower body cavity and secured by suturing. The wound was closed with metal clips. One hour before the operation, the animals were anesthetized with a mixture of ketamine (43 mg/kg bw) and xilacin (8.7 mg/kg bw) that was injected intraperitoneally. In addition, they were also intramuscularly injected with a pain reliever, buprenorphine (0.05 mg/kg bw). As a post-operative, the same dose of buprenorphine was also administered every 8 h for 2 days. Animals were allowed to recover for 10 days before starting the study and were housed in a temperature-controlled room (23° C.) and light/dark cycle (8:00 am-8:00 pm). After the operation recovery period and keeping the water and feed conditions ad-libtum, the animals were divided into two groups. One group received vehicle (negative control consisting of 20% condensed milk) and the other received UVC wine lees lyophilisate (125 mg/kg bw) dissolved in vehicle. The dose 125 mg/kg bw is equivalent to the same dose at which lees were tested in the acute administration study (Example 3). The volume administered to both groups of animals was 1.5 mL. Treatments (vehicle or lees) were performed daily for 5 weeks.

For SBP measurement, rats were individually placed in plastic cages that were placed on top of the receptors, and blood pressures were recorded 6 to 7 h after administration of the treatments, 3 times per week (every two days). The measurement was made during that hour, 500 times per second. The animals' SBP was also recorded the day before treatment (week 0) to determine their initial values. The SBP results obtained in each group being analyzed were represented as mean±ESM (n=6 animals of 3 weekly measurements). A t-Student test was used to determine differences between treatments in each study week using the SPSS statistical program (IBM SPSS Statistics software version 20.0). The difference was considered significant for values of p<0.05.

Table 3 shows the SBP of the animals during the study. Prior to the treatments, the animals had an SBP of 176±3 mmHg, indicating that the animals had hypertension. The SBP of the animals treated with the vehicle increased as the 5 weeks of treatment elapsed, observing in the last week of treatment an increase of approximately 20 mmHg with respect to the initial one. However, when the animals were treated with the lyophilized UVC wine lees, there were no changes in the SBP, maintaining the initial values of SBP at the end of the experiment. Specifically, significant differences were observed in the SBP of the animals treated with the lees compared to that of the animals treated with the vehicle at week 4 and 5 of the treatment. Fact that indicates that the treatment with the lees prevents the increase in blood pressure.

TABLE 3
SBP in hypertensive rats, recorded at 6 h post-administration

| | SBP (mmHg)* | | | | | |
|---|---|---|---|---|---|---|
| Treatment | Week 0 | Week 1 | Week 2 | Week 3 | Week 4 | Week 5 |
| Vehicle | 176 ± 3 | 183 ± 2 | 188 ± 2 | 189 ± 1 | 195^{a} ± 1 | 203^{a} ± 6 |
| Lyophi- | 176 ± 3 | 176 ± 1 | 184 ± 2 | 184 ± 2 | 180^{b} ± 2 | 180^{b} ± 3 |
| lized lees | | | | | | |

Different letters indicate significant differences between treatments at a given week (t-Student test).

### Example 6: Effect of Acute Ingestion of UVC Wine Lees on the Blood Pressure of Normotensive Rats

The present example describes the effect of UVC wine lees in Wistar Kyoto (WKY) rats, which are the SHR normotensive control, in order to rule out a possible hypotensive effect (undesirable effect).

For the study, male WKY rats 17-20 weeks old and weighing between 326 and 370 g, from Charles River Laboratories Espana S.A. were used. Initial values of SBP and DBP were 119.1±4.2 and 87.9±8.8 mmHg, respectively, which indicated their normal pressure state. Stalling conditions and methodology used to determine SBP are described in Example 3.

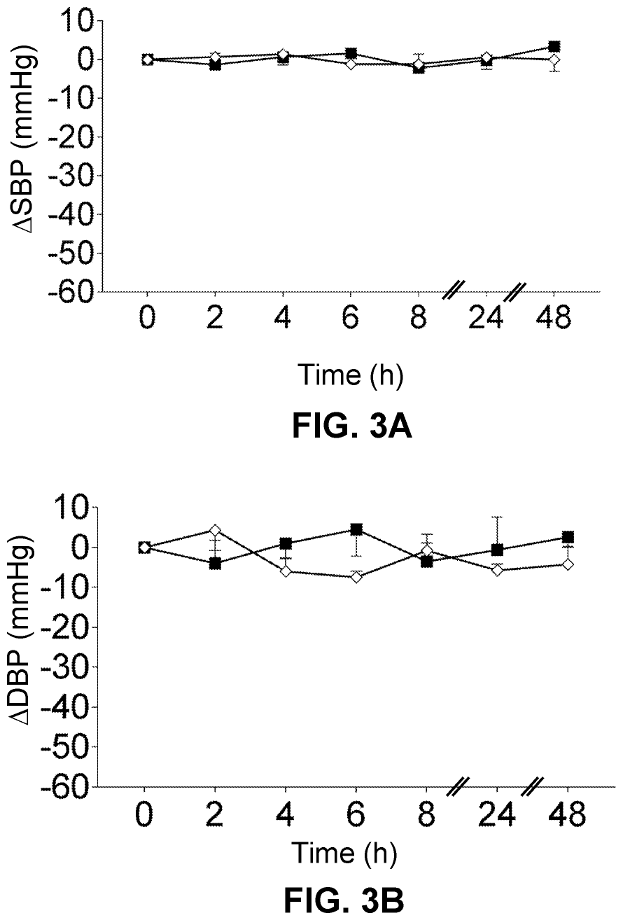

The administration of a single dose of lyophilized UVC wine lees (5 mL/kg bw) did not modify SBP or DBP values in the animals during the experiment (FIGS. 3A and B, respectively). BP values were significantly similar to those showed by the animals that ingested water.

These results allow us to rule out possible undesirable effects of the UVC wine lees on BP of normotensive subjects and indicates that the lees only exert their antihypertensive action when there is a pathological state of hypertension as shown in Examples 3 and 4.

### Example 7: Procedure for Obtaining Hydrolysates from Wine Lees

In a round bottom plastic tube, 20 mL of UVC wine lees were added, with evident antihypertensive activity (Examples 3 and 4). The pH was adjusted to 7 or 4 with 5M NaOH (depending on the enzyme used). Subsequently, the enzyme preparation was added in a volume of 0.5 mL. The hydrolysis was carried out with the enzymes: Alcalase® 2.4 L, Flavourzyme® 1000 L and a mixture of Alcalase® 2.4 L and Protamex® (Alcalase®+Protamex®). These enzymes were added at an enzyme/protein concentration of 0.4 AU (Alcalase®, and Alcalase®+Protamex®) or 80 LAPU (Flavourzyme®). Since enzymes show different activities (2.4; 1.5 Anson Units (AU)/g and 1000 Leucine Aminopeptidase Units (LAPU)/g, respectively), the added volume of enzyme varied depending on the enzyme and concentration of enzyme/protein tested. For this reason, the final volume was adjusted with distilled water so that they all underwent the same dilution (2.5 mL). The Protamex® enzyme, sold in solid state, was reconstituted in distilled water to the desired concentration before adding it to the lees.

At the time of carrying out these tests, Protamex® corresponds to the proteolytic enzymes from Bacillus licheniformis and Bacillus amyloliquefaciens (E.C. 3.4.21.62 and 3.4.24.28); Flavourzyme® is an aminopeptidase from Aspergillus oryzae (E.C. 3.4.11.1); and Alcalase® 2.4 L, also called Carlsberg subtilisin, is a serine protease from Bacillus licheniformis (E.C. 3.4.21.62); all of them from Novozyme, Nordisk. Hydrolysis was carried out at 25° C. with constant stirring at 200 rpm in orbital, for 2 h at a pH of 7 (Alcalase®, Alcalase®+Protamex®) or 4 (Flavourzyme®). After the hydrolysis, the enzymes were inactivated using different methods: by heat (in a water bath at 85° C. for 10 min) or by lowering the pH to pH 3. Finally, all the samples were centrifuged at 2,950×g, 15 min, 4° C. for the elimination of non-soluble compounds. The supernatants obtained correspond to the hydrolysates of the present invention, which were frozen at −20° C. until further analysis.

ACEI activity was analyzed according to the method described in Example 2. Table 4 shows the results obtained from ACEI activity for hydrolysates, diluted 1/256. Also, the ACEI activity of the lees (after being centrifuged at 2,950×g, 15 min, 4° C.) without being hydrolysed subjected to the same procedure as the hydrolysates, except for the addition of the enzyme, was determined. The dilution produced by the addition of the volume of enzyme in the hydrolysates and/or volume of HCl used to lower the pH of the sample was taken into account.

TABLE 4
ACEI activity of the protein hydrolysates obtained from
the UVC wine lees. Data are expressed as mean (n = 3).

| | | | ACEI | ACEI |
|---|---|---|---|---|
| | | | activity | activity |
| hydrolysate | Enzyme | Inactivation | (%)* | (IC₅₀, μL) |
| 1 | Alcalase ® | 85° C., 10 min | 45.85 | 0.19 |
| 2 | Alcalase ® + | 85° C., 10 min | 47.71 | 0.18 |
| | Protamex ® | | | |
| 3 | Flavourzyme ® | pH 3 | 53.59 | 0.16 |

*Dilution of hydrolysates used: 1/256

All the hydrolysates showed an ACEI activity higher than that of the starting lees (38.21 and 35.68% for the treatment of enzymatic inactivation by heat and pH, respectively). These results indicate that, although the lees by themselves have ACEI activity due to the fact that they consist of bioactive compounds, enzymatic hydrolysis produces peptides with ACE inhibitory power that cause these hydrolysates to have a better ACEI activity compared to the starting lees.

In addition, the minimum amount to inhibit 50% of the ACE of the hydrolysates was determined, being in all cases lower than 0.20 μL.

The study was continued with “hydrolysate 3”, which is the one that showed the lowest IC₅₀.

### Example 8: Antihypertensive Activity in Hypertensive Rats of “Hydrolysate 3”

Male SHR rats were used to determine the possible antihypertensive effect of this hydrolysate. The age, stalling conditions of the rats and the BP measurement technique were the same as those described in Example 3.

The antihypertensive effect of the “hydrolysate 3” was studied for 72 h (0, 2, 4, 6, 8, 24, 48, and 72 h), after its administration by intragastric tube. As in Example 3, negative and positive BP controls were performed, obtained after administration of water and captopril (50 mg/kg bw) respectively, in a volume of 1.5 mL per animal. The antihypertensive activity of the wine lees used to prepare “hydrolysate 3” and “hydrolysate 3” was evaluated, both at a dose of 5 mL/kg bw. The lees used in these tests were from a different batch than the one used in Example 3.

BP measurements were pooled and the mean±ESM (n=6 per group) was obtained. The same analysis of variance was used as in Example 3.

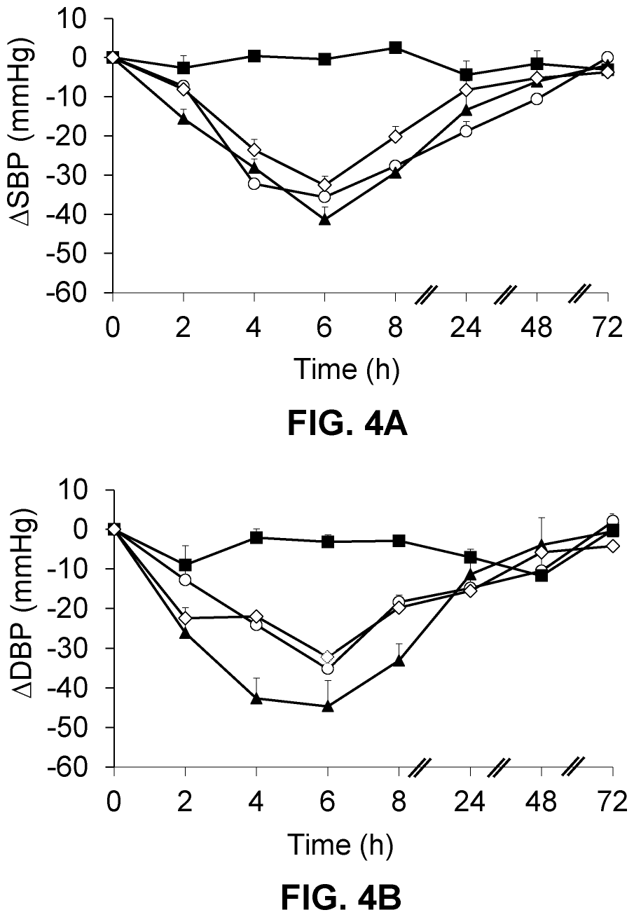

As shown in FIG. 3, captopril produced a decrease in SBP (FIG. 4) in SHR rats after 2 h of its administration, being maximum at 6 h of its consumption (−43 mm Hg in both cases). The same pattern of SBP drop occurred after treatment with unhydrolysed UVC wine lees. The maximum decrease of SBP produced by the hydrolysate was observed at 4 h post-administration (−32 mm Hg), remaining at the same SBP levels until 8 h. This decrease was significantly similar to that produced by captopril except for that observed at 2 h post-administration (greater in captopril). In later hours, the SBP began to rise slowly until 72 h when the basal levels were reached. Indicative fact that the hydrolysate was losing its antihypertensive effect. It is noteworthy that the antihypertensive effect of the hydrolysate was much longer than that of captopril (antihypertensive drug), which no longer exerted any significant effect 48 h after administration, while the hydrolysate produced a decrease in SBP of −12 mm Hg at that time.

In addition, it should be indicated that the pattern of SBP decrease produced by the administration of the hydrolysate and the lees (same lees as those used to make the hydrolysate) was different. At 4 h post-administration the hydrolysate reached its maximum activity (−32 mmHg) while the lees produced only a drop in SBP of −23 mmHg. Furthermore, the effectiveness of the hydrolysate lasted much more longer. At 24 h post-administration, the hydrolysate still produced a powerful antihypertensive effect (−20 mmHg) while, in the animals treated with the UVC wine lees, the antihypertensive effect had already disappeared. This different effect of both products (wine lees and hydrolysate) confirms that during the hydrolysis process described in the present invention, the release of peptides with antihypertensive activity occurs, which were not present in the starting lees.

Lastly, it should be noted that, as previously mentioned, the lees used in this study were from a different batch than that used in Example 3. The results show that the administration of different batches (campaign) of lees obtained in the elaboration of UVC wine to hypertensive animals produce an antihypertensive effect, indicating the reproducibility of the effect and of obtaining the ingredient.

### Example 9: Isolation, Identification and Synthesis of Peptides with ACEI Activity

Analytical (ThermoFisher Scientific nano-HPLC Easy-LC) and semi-preparative (Agilent Technologies 1260 series) high efficacy liquid chromatography (HPLC) equipment, as well as a mass spectrometry kit (nanoLC-Orbitap-MS/MS from Thermo Scientific) that allowed the sequencing of the peptides, were used to carry out this work. The steps mentioned below were performed.

9.A. Obtaining “Hydrolysate 3” and the 3,000 Daltons Minor Protein Fraction

Following the procedure described in Example 7, 250 mL of “hydrolysate 3” were obtained from the UVC wine lees. The supernatant obtained again was centrifuged at 3,000×g, 180 min, 4° C. in filter devices (Centripep, Amicon Inc) with a hydrophilic membrane of 3,000 Da pore size. The permeate obtained (fraction less than 3,000 Da) was collected, lyophilized and stored at −20° C. until its subsequent fractionation.

9.B. Reverse Phase HPLC Fractionation on a Semi-Preparative Scale

Fractionation of the fraction of less than 3,000 Da obtained from “hydrolysate 3” was carried out using semi-preparative HPLC. The methodology and equipment used was that described by Bravo et al (Novel antihypertensive peptides derived from chicken foot proteins. Mol. Nutr. Food Res. 2019, 63, 1801176), except for the gradient used to separate the peptides. This consisted of: 0 to 23.5% B in 39.17 min, 23.5-90% B in 9.83 min and 90-0% B in 1 min.

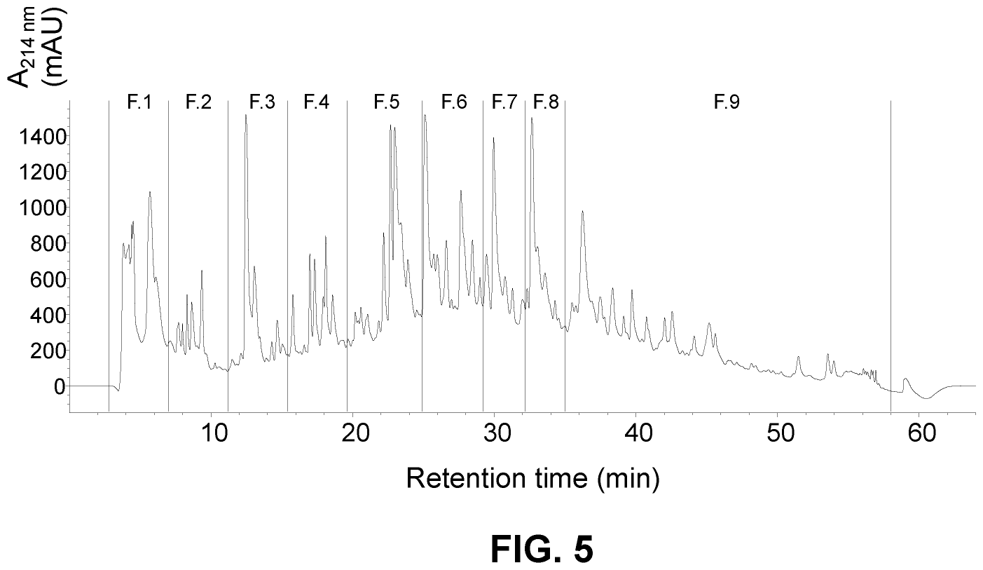

9 different fractions were collected (FIG. 5), called F.1-F.9, to facilitate the identification of the peptides, which were lyophilized and reconstituted in Milli Q water.

9.C. Identification of “Hydrolysate 3” Peptides by Tandem Mass Spectrometry (MS/MS)

To determine the peptide sequence of the peptides, contained in the fractions, an analysis was carried out by mass spectrometry with a nanoLC-(Orbitrap) MS/MS equipment (LTQ-Orbitrap Velos Pro mass spectrometer (Thermo Fisher, San Jose, Calif., USA). The protocol described by Bravo et al. (“Novel antihypertensive peptides derived from chicken foot proteins”. Mol. Nutr. Food Res. 2019, 63, 1801176) was followed. All MS and MS/MS samples were analyzed using the Mascot search engine (Version 2.5) using three different nodes: one with the Vitis vinifera proteome (29907 entries) from the uniprot database, the second with the limited Swiss-Prot database from the taxonomy of fungi (33712 entries) and the last one with the common contaminants for proteomic applications (247 entries). Thus, the 6 peptides from Table 6 were identified.

9.D. Synthesis of the Peptides and Determination of their ACEI Activity

Once the peptides were identified, their ACEI activity was determined in order to identify which of the peptides could be responsible for the bioactivity of the hydrolysate. For this, the peptides were chemically synthesized at Caslo ApS (Lyngby. Denmark) by the Fmoc method in solid phase with a synthesizer (model 431A; Applied Biosystems Inc., Uberlingen, Germany). Table 5 shows the purity of the synthesized peptides, determined by Caslo ApS by analytical-scale reverse phase HPLC and mass spectrometry using an Esquire-LC kit (Bruker Daltonik GmbH. Bremen. Germany).

These synthetic peptides were diluted in Milli Q water and the ACEI activity was determined following the method described in Example 2 (Table 5). The concentration of the peptides of which the ACEI activity (%) was determined was 0.83 mg/m L.

TABLE 5
Peptides, purity and ACEI activity (%)

| | | | | | ACEI |
|---|---|---|---|---|---|
| | | SEQ ID | | Purity | activity |
| Peptides | Sequence | No. | MW | (%) | (%) |
| P1 | FKTTDQQTRTTVA | 1 | 1,496.65 | 97.8 | 27.0 ± 5.6 |
| P2 | NPKLVTIV | 2 | 883.11 | 99.2 | 48.5 ± 9.1 |
| P3 | TVTNPARIA | 3 | 942.09 | 96.4 | 76.4 ± 9.2 |
| P4 | PAGELHP | 4 | 719.80 | 99.4 | 72.3 ± 11.5 |
| P5 | LDSPSEGRAPG | 5 | 1,085.15 | 96.9 | 95.4 ± 2.2 |
| P6 | LDSPSEGRAPGAD | 6 | 1,271.32 | 98.5 | 2.4 ± 0.9 |

All the peptides tested had a purity greater than 96%. The P3, P4 and P5 peptides were those that showed the highest in vitro activity, showing an inhibitory activity of more than 70%.

### Example 10: Antihypertensive Activity in Rats of the Peptides Identified in “Hydrolysate 3” Obtained from Wine Lees Made with UVC

The present example describes the modification of SBP produced by the acute administration of the P1-P6 peptides (SEQ ID No. 1-6, respectively).

For the evaluation of the possible acute antihypertensive effect of the peptides, a study in SHR rats was carried out, provided by Charles River Laboratories Espana S.A. The rats' age, stalling conditions and BP measurement technique were the same as those described in Example 3. The initial SBP of the animals was 204±2 mmHg. A negative control (water) and a positive control (captopril) were also included during the study. The peptides were administered intragastrically at a single dose of 10 mg/kg bw and captopril at 50 mg/kg bw, diluted in water. BP measurements were pooled and the mean±ESM was obtained (n=6 per group). The same analysis of variance was used as in Example 3.

Table 6 shows the variation of the SBP of the animals treated with the different compounds from the moment before their administration (0 h) to 48 h after their administration.

TABLE 6
ΔSBP produced in spontaneously hypertensive rats after administration
of the six peptides identified in the “hydrolysate 3”.

| | ΔSBP (mmHg)* | | | | | |
|---|---|---|---|---|---|---|
| Treatment | 2 h | 4 h | 6 h | 8 h | 24 h | 48 h |
| Water | −1 ± 3 | −1 ± 3 | 1 ± 2 | −3 ± 3 | −5 ± 4 | −2 ± 2 |
| Captopril | −19 ± 6 | −34 ± 4 | −44 ± 4 | −45 ± 3 | −22 ± 6 | −7 ± 4 |
| Peptide P1 | −12 ± 2 | −15 ± 4 | −23 ± 5 | −24 ± 3 | −13 ± 3 | −1 ± 3 |
| Peptide P2 | −9 ± 6 | −13 ± 4 | −19 ± 5 | −14 ± 3 | −5 ± 4 | −5 ± 3 |
| Peptide P3 | −2 ± 4 | −14 ± 4 | −16 ± 2 | −14 ± 4 | −5 ± 3 | −2 ± 3 |
| Peptide P4 | −7 ± 3 | −5 ± 4 | −6 ± 5 | −5 ± 4 | −3 ± 7 | −2 ± 3 |
| Peptide P5 | −10 ± 4 | −12 ± 5 | −18 ± 3 | −18 ± 3 | −3 ± 4 | −1 ± 4 |
| Peptide P6 | −11 ± 2 | −16 ± 4 | −24 ± 3 | −22 ± 4 | −2 ± 4 | −5 ± 4 |

*Decrease calculated as: SBP of the different hours − SBP of 0 h. Data represented as Mean ± ESM (n = 6).

As shown in Table 6, the SBP of the animals that were administered water (negative control) did not vary significantly throughout the study. However, the SBP of the animals treated with captopril (positive control) decreased at 2 h after its administration (−19 mmHg), being maximum at 4-6 h after its administration. Its effect lasted until 24 h after its administration, and SBP decreases of −22 mmHg were still observed. As for the peptides, treatment with the peptides P1, P2, P3, P5 and P6 produced a decrease in SBP, being of more than 10 mmHg. It disappeared 24 or 48 h after their administration. Regarding P4, its administration to SHR did not show an antihypertensive effect. The SBP value is considered a good indicator of cardiovascular risk and, in fact, a reduction in it of 10 mmHg results into a significant reduction in the risk of suffering CVD.

The peptides that produced the greatest effectiveness were the peptides P1 and P6. Specifically, the acute administration of the peptides P1 and P6 to the animals produced a powerful antihypertensive effect, reducing the SBP of the animals from the 2 h post-administration and reaching maximum values of up to −23 and −24 mmHg, respectively, at 4 h post-administration. Furthermore, they were able to maintain this reduction at least until 8 h after their administration. No significant differences were observed with the effect produced by the captopril drug.

The P2, P3 and P5 peptides also produced a decrease in the SBP, showing significant differences with both the SBP presented by the animals treated with water. Fact that shows its antihypertensive effect. The effect was maximum at 6 h post-administration, producing a decrease in SBP of between −16 and −19 mmHg.

Thus, it is demonstrated that peptides of the present invention have an antihypertensive effect in vivo.

1. (canceled)

2. (canceled)

3. (canceled)

4. (canceled)

5. (canceled)

6. (canceled)

7. (canceled)

8. (canceled)

9. (canceled)

10. Wine lees hydrolysate comprising a peptide selected from the group consisting of SEQ ID No. 1-SEQ ID No. 6, and combinations thereof.

11. Wine lees hydrolysate according to claim 10 comprising a peptide of sequence SEQ ID No. 1 and/or a peptide of sequence SEQ ID No. 6.

12. Wine tees hydrolysate according to claim 10, wherein the wine lees are wine lees from grapes of the Cabernet variety and/or Tempranillo variety.

13. Wine lees hydrolysate, wherein the wine lees are wine lees of grapes of the Cabernet variety and/or Tempranillo variety.

14. Wine lees hydrolysate according to claim 10 or 13, wherein the hydrolysate has ACE inhibitory (ACEI) activity to vitro with an IC₅₀ lower than or equal to 0.25 μL.

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. Wine lees hydrolysate according to claim 10 or 13, formulated with one or more vehicles, diluents, solvents or additives in the form of a nutraceutical composition, pharmaceutical composition, food composition, medicament, food supplement, beverage or food product.

34. Method for obtaining a hydrolysate according to claim 10, characterized in that it comprises the following steps:
a) Enzymatic hydrolysis o wine lees with one or more enzymes selected from the group consisting of protease, serine protease or aminopeptidase, under conditions suitable for the enzyme/s;
b) Inactivation of the enzyme.

35. Method according to claim 34, wherein the wine lees are from grapes of Cabernet variety and/or Tempranillo variety.

36. Method accordion to claim 34 or 35, wherein the protease is from Bacillus amyloliquefaciens, the serine protease is from Bacillus licheniformis, and the aminopeptidase is from Aspergillus oyzae.

37. Method according to claim 34, wherein step a) is carried out from 1 to 24 hours.

38. Method according to claim 34, further comprising the following steps:
c) Evaluation of the ACEI activity; and
d) Selection of a hydrolysate with an ACEI activity in vitro with IC₅₀ lower than or equal to 0.25 μL.

39. Isolated peptide the sequence of which is selected from the group consisting of SEQ ID No. 1-SEQ TD No, 6.

40. The peptide according to claim 39, wherein the sequence is SEQ ID No. 1 or SEQ ID No. 6.

41. The peptide according to claim 39 or 40, formulated with one or more vehicles, diluents, solvents or additives in the form of a nutraceutical composition, pharmaceutical composition, food composition, medicament, food supplement, beverage or food product.

42. Method for treating or preventing a disease in which the wine lees hydrolysate defined in claim 10, 13 or 33, or the peptide as defined in claim 39 or 41, or wine lees from grapes of the Cabernet variety and/or Tempranillo variety or their supernatant, is administered to a subject in need thereof.

43. Method according to claim 42, wherein the disease is selected from hypertension, a cardiovascular disease (CVD) associated with hypertension or a disease treated with Angiotensin Converting Enzyme (ACE) inhibitors selected from the group consisting of heart failure, coronary artery disease, diabetes, and chronic kidney disease.

44. Method according to claim 43, wherein the wine lees hydrolysate, the peptide or the wine lees, is administered as a coadjuvant.

45. Method according to claim 42 or 43, wherein the sequence of the peptide is SEQ ID No. 1 or SEQ ID No. 6.
